SEQUENCE LISTING (53 sequences)

SEQ ID NO: 1 - protein, 21 aa, Homo sapiens
CEEMEEGYTQCSQFLYGVQEK

SEQ ID NO: 2 - protein, 26 aa, Homo sapiens
MVTSLACGNGVCGCSPGGDTDTQEAK

SEQ ID NO: 3 - protein, 24 aa, Homo sapiens
KGADHSSAPPADGDDEEMMPGHHL

SEQ ID NO: 4 - protein, 23 aa, Homo sapiens
VTPQEEADSDVGEEPDSENTPQK

SEQ ID NO: 5 - protein, 26 aa, Homo sapiens
GQCPPPPGLPCPCTGVSDCSGGTDKK

SEQ ID NO: 6 - protein, 20 aa, Homo sapiens
WMLQSMAEWHCQHQEQGMLK

SEQ ID NO: 7 - protein, 22 aa, Homo sapiens
DDEPDPLILEENDVDNMATNNK

SEQ ID NO: 8 - protein, 21 aa, Homo sapiens
VNISCKASQDIDDDMNWYQQK

SEQ ID NO: 9 - protein, 21 aa, Homo sapiens
DWILYAEQDSNHCFISTVECK

SEQ ID NO: 10 - protein, 23 aa, Homo sapiens
QLLEDSTSDEDRSSSSSSEGKEK

SEQ ID NO: 11 - protein, 26 aa, Homo sapiens
SEDGTPAEDGTPAATGGSQPPSMGRK

SEQ ID NO: 12 - protein, 23 aa, Homo sapiens
SQDSPEISSLCQGEEATPRHSDK

SEQ ID NO: 13 - protein, 25 aa, Homo sapiens
AVTDTHENGDLGTASETPLDDGASK

SEQ ID NO: 14 - protein, 24 aa, Homo sapiens
MQSSSSLSSGESAQVSTENNELTK

SEQ ID NO: 15 - protein, 24 aa, Homo sapiens
EGCEPQSASPQSKEQQGDARGSPK

SEQ ID NO: 16 - protein, 22 aa, Homo sapiens
TELCENVDPNITSEDLSLHKED

SEQ ID NO: 17 - protein, 8 aa, Homo sapiens
CEEMEEGY

SEQ ID NO: 18 - protein, 6 aa, Homo sapiens
TQCSQF

SEQ ID NO: 19 - protein, 5 aa, Homo sapiens
GVQEK

SEQ ID NO: 20 - protein, 5 aa, Homo sapiens
MVTSL

SEQ ID NO: 21 - protein, 21 aa, Homo sapiens
ACGNGVCGCSPGGDTDTQEAK

SEQ ID NO: 22 - protein, 23 aa, Homo sapiens
GADHSSAPPADGDDEEMMPGHHL

SEQ ID NO: 23 - protein, 23 aa, Homo sapiens
VTPQEEADSDVGEEPDSENTPQK

SEQ ID NO: 24 - protein, 9 aa, Homo sapiens
GQCPPPPGL

SEQ ID NO: 25 - protein, 16 aa, Homo sapiens
PCPCTGVSDCSGGTDK

SEQ ID NO: 26 - protein, 6 aa, Homo sapiens
QSMAEW

SEQ ID NO: 27 - protein, 10 aa, Homo sapiens
HCQHQEQGML

SEQ ID NO: 28 - protein, 7 aa, Homo sapiens
DDEPDPL

SEQ ID NO: 29 - protein, 13 aa, Homo sapiens
EENDVDNMATNNK

SEQ ID NO: 30 - protein, 6 aa, Homo sapiens
VNISCK

SEQ ID NO: 31 - protein, 11 aa, Homo sapiens
ASQDIDDDMNW

SEQ ID NO: 32 - protein, 3 aa, Homo sapiens
QQK

SEQ ID NO: 33 - protein, 9 aa, Homo sapiens
AEQDSNHCF

SEQ ID NO: 34 - protein, 6 aa, Homo sapiens
STVECK

SEQ ID NO: 35 - protein, 18 aa, Homo sapiens
EDSTSDEDRSSSSSSEGK

SEQ ID NO: 36 - protein, 25 aa, Homo sapiens
SEDGTPAEDGTPAATGGSQPPSMGR

SEQ ID NO: 37 - protein, 10 aa, Homo sapiens
SQDSPEISSL

SEQ ID NO: 38 - protein, 9 aa, Homo sapiens
CQGEEATPR

SEQ ID NO: 39 - protein, 4 aa, Homo sapiens
HSDK

SEQ ID NO: 40 - protein, 11 aa, Homo sapiens
AVTDTHENGDL

SEQ ID NO: 41 - protein, 8 aa, Homo sapiens
GTASETPL

SEQ ID NO: 42 - protein, 6 aa, Homo sapiens
DDGASK

SEQ ID NO: 43 - protein, 7 aa, Homo sapiens
MQSSSSL

SEQ ID NO: 44 - protein, 15 aa, Homo sapiens
SSGESAQVSTENNEL

SEQ ID NO: 45 - protein, 13 aa, Homo sapiens
EGCEPQSASPQSK

SEQ ID NO: 46 - protein, 7 aa, Homo sapiens
EQQGDAR

SEQ ID NO: 47 - protein, 4 aa, Homo sapiens
GSPK

SEQ ID NO: 48 - protein, 3 aa, Homo sapiens
TEL

SEQ ID NO: 49 - protein, 13 aa, Homo sapiens
CENVDPNITSEDL

SEQ ID NO: 50 - protein, 21 aa, Carassius auratus
SPANAQITRKRHKINSFVGLM

SEQ ID NO: 51 - protein, 9 aa, Carassius auratus
SPANAQITR

SEQ ID NO: 52 - protein, 8 aa, Carassius auratus
INSFVGLM

SEQ ID NO: 53 - protein, 10 aa, Carassius auratus
HKINSFVGLM

(19) United States
(12) Patent Application Publication
(10) Pub. No.: US 2009/0256068 A1
(43) Pub. Date: Oct. 15, 2009

(54) SOLUTION FRAGMENTATION SYSTEMS AND PROCESSES FOR PROTEOMICS ANALYSIS

(76) Inventors: Konstantinos Petritis, Richland, WA (US); Richard D. Smith, Richland, WA (US)

Correspondence Address: BATTELLE MEMORIAL INSTITUTE;ATTN: IP SERVICES, K1-53, P. O. BOX 999, RICHLAND, WA, 99352, US

(21) Appl. No.: 12/100,905

(22) Filed: Apr. 10, 2008

(51) Int. Cl. B01D 59/44; C07K 1/14; C12P 21/06

(52) U.S. Cl. 250/282; 530/344; 435/68.1

(57) ABSTRACT

A solution-phase digestion process is described. Intact proteins are digested to obtain parent peptides, which are separated and subsequently mass analyzed. Individual parent peptides are digested to obtain daughter peptides, which are also subsequently mass analyzed. Accurate mass data obtained from mass analysis of both parent and daughter peptides are correlated with separations data obtained during separation of the parent peptides to provide peptide identification. The process is expected to provide unique peptides by which to identify intact proteins in a sample without need for MS/MS gas-phase fragmentation.

This invention was made with Government support under Contract DE-AC06-76RLO1830 awarded by the U.S. Department of Energy. The Government has certain rights in the invention.

### FIELD OF THE INVENTION

The present invention relates generally to fragmentation and analysis of proteins. More particularly, the invention is a system and a process for fragmentation of proteins “in solution”. The invention finds application in, e.g., proteomics analysis for identification of proteins.

### BACKGROUND OF THE INVENTION

Recent developments in mass spectrometry are enabling proteomics analysis for identification of biological molecules. Speed, specificity, and sensitivity of mass spectrometry make it especially attractive for rapid characterization and identification of proteins. Protein identification typically involves comparing mass data and information obtained from mass spectrometry analysis of chemically- or proteolytically-derived peptide ions, with characteristic peptide masses (so-called peptide “fingerprints”) compiled in database searches to identify the protein. Protein identification can also be accomplished by obtaining mass data of individual peptides using, e.g., tandem mass spectrometry (MS/MS), followed by interrogation of product ion spectra compiled, e.g., in such worldwide web databases as PROSPECTOR, (prospector.ucsf.edu); PROFOUND (65.219.84.5/Proteinld.html or prowl.Rockefeller.edu); and MASCOT (www.matrixscience.com) that provide for protein sequence analysis. Protein sequence information can also be extracted from databases using such constraints as, e.g., experimentally observed mass ranges; or isoelectric point data for intact proteins which can then be digested in silico into corresponding peptides that provide associated theoretical peptide masses. Experimentally determined peptide masses can then be compared to the theoretical peptide masses. Subsequent ranking of proteins can then be based on numbers of peptides for a given protein in the database that match with experimental peptide masses. While this approach is amenable to analysis of simple protein mixtures, mass fingerprinting is not generally suited to analysis of peptides from complex protein mixtures, as peptides from many different proteins are present that complicates assigning individual peptides to the correct proteins. And, databases often contain incomplete information by which to identify a protein, e.g., in a complex mixture. In practice, identification of large proteins and peptides using conventional MS/MS techniques remains difficult because large proteins and peptides are poorly ionized; sufficient fragmentation is not obtained in the gas phase; or, because loss of structural information prior to analysis leads to loss of sensitivity needed for protein and peptide identifications. Accordingly, new processes are needed that provide sufficient fragmentation for identification of large proteins and peptides for high throughput and quantitative proteomics analyses.

### SUMMARY OF THE INVENTION

The present invention includes a system for fragmentation of proteins in solution (termed “in-solution” fragmentation) that includes: a fragmentation (digestion) stage, where intact proteins and polypeptides in a sample are cleaved into parent peptides of a preselected size; a separations stage, where parent peptides are separated to obtain individual parent peptides or groups of parent peptides; at least one additional in-solution fragmentation (digestion) stage, where separated parent peptides are fragmented (digested) into daughter peptides with a size that is smaller than the parent peptides; and an analysis stage, where parent peptides and corresponding daughter peptides are analyzed for identification of the sample proteins. The present invention also includes a process for fragmenting proteins in solution that includes the steps of: fragmenting (digesting) a protein in solution or in gel to obtain parent peptides; separating the parent peptides to obtain individual parent peptides or groups of parent peptides; digesting the individual parent peptides or the groups of parent peptides at least partially in solution or in gel to obtain at least a quantity of daughter peptides. The present invention also includes a process for fragmenting proteins in solution (termed “in-solution” fragmentation) that includes the steps of: fragmenting a protein in solution or in gel to obtain parent peptides; separating the parent peptides to obtain individual parent peptides or groups of parent peptides; fragmenting (digesting) a preselected portion of individual parent peptides or groups of parent peptides in solution to obtain daughter peptides for same. The daughter peptides have a size that is smaller than the parent peptides. Daughter peptides are typically smaller in size than the parent peptides from which they are derived. At least one preselected portion or fraction of each individual parent peptides or groups of parent peptides is retained intact for subsequent analysis; and analyzing individual parent peptides, groups of parent peptides, and corresponding daughter peptides for more accurate identification of the sample proteins. Fractions containing preselected quantities of each individually separated parent peptide or group of parent peptides, and daughter peptides can be subjected to mass analysis in various ways. In one embodiment, mass analysis of each parent peptide or group of parent peptides in at least one fraction, with corresponding mass analysis of daughter peptides derived from in solution fragmentation of parent peptides in another fraction is done simultaneously (e.g., in different mass analyzers) that yields accurate mass data for both parent and daughter peptides with an identical analysis time profile. In another embodiment, mass analysis of parent and daughter peptides is done in a single analyzer in succession, e.g., in conjunction with a dual channel ion funnel. Daughter peptides, since they are derived from parent peptides following separation of the parent peptides, have elution profiles that match with the parent peptides, which provides ability to correlate accurate mass data for individual parent peptides with mass data for the corresponding daughter peptides, that provides more accurate identification of the daughter peptides, parent peptides, and proteins and polypeptides in the sample. In-solution fragmentation processes of the invention are not limited to selected proteins. Proteins in a sample can include de novo proteins. Proteins in a sample can also be synthesized in vitro. Proteins can also be in-silico proteins. Proteins in a sample can include human proteins, animal proteins, insect proteins, mammalian proteins, cellular proteins, bacterial proteins, proteins that contain nucleic acids (e.g., RNA and DNA), and other biological proteins, including combinations of the listed types. Parent peptides generated by digestion can be separated using any liquid separations process (e.g., a liquid chromatography process) or separations devices (e.g., a separations column such as a liquid chromatography separations column). Separation of parent peptides may be accomplished in online or in offline operations, using LC columns in concert with various stationary phases. Separation of peptides may also be accomplished using lab-on-a-chip and multiplate separation processes and devices; high-efficiency multidimensional separation processes and devices, microseparations processes and devices including, e.g., microfluid and microcolumn separation processes and devices; Electrophoresis, Capillary Electrophoresis (CE), Dielectrophoresis (DEP), Capillary Isoelectric Focusing, Gel separations in one or more dimensions, including, but not limited to, e.g., 2-D Gel Electrophoresis, and Sodium Dodecyl Sulfate Polyacrylamide Gel Electrophoresis (SDS-PAGE); and like separation processes or devices. Peptides may also be separated to obtain elution data and elution profiles that include, but are not limited to, e.g., molecular weight data; isoelectric point data; elution time data; retention time data; and peptide predictions for peak elution times for parent and daughter peptides; and like parameters. Preselected quantities of separated parent peptides are portioned into at least a first and second fraction (in offline operation) or analysis stream (in online operation) using a stream splitter or equivalent stream splitting means. At least one fraction containing individual parent peptides is introduced in succession to a digestion stage and digested enzymatically with enzymes including, e.g., trypsin, chymotrypsin, pepsin, and like proteases. Parent peptides are digested to obtain daughter peptides using orthogonal enzymes, i.e., different enzymes from those used in the prior digestion of proteins that yield parent peptides. In other embodiments, following post column separation, parent peptides can be digested to obtain daughter peptides in a digestion stage in one or more flow paths that contain one or more different enzymes in succession. In other embodiments, parent peptides can be digested using immobilized enzymes. Configurations are not limited. Daughter peptides provide additional structural information by which daughter and parent peptides can be identified. Daughter peptides have a molecular weight that is at or below the molecular weight of the parent peptide from which they are derived. Daughter peptides preferably have molecular weights in the range from about 300 Daltons to about 6,000 Daltons, but are not limited thereto. More preferably, daughter peptides have a molecular weight up to about 1,500 Daltons. In-solution fragmentation described herein provides for analysis of parent peptides, and/or daughter peptides without need of a fragmentation step in the gas phase of a mass analyzer. In one analysis process involving a dual mass analyzer configuration, parent peptides in a first analysis stream or fraction and daughter peptides in a second analysis stream or fraction can be concurrently analyzed, which provides accurate mass data for both parent peptides and daughter peptides with equivalent analysis times; elution profiles are also identical permitting alignment and correlation of accurate mass data and elution data for both parent and daughter peptides for identification of the peptides. In an alternate process, parent and daughter peptides can be analyzed in a single mass analyzer, e.g., serially. Analysis of at least a first and a second analysis stream in an MS analyzer can include an MS/MS analysis of at least one of the analysis streams. The apex of elution peaks for daughter peptides generated in the digestion of parent peptides substantially matches an apex of elution peaks from parent peptides generated from the digestion of sample proteins, such that daughter peptides and/or fragments can be aligned and assigned to individual parent peptides in combination with additive measures, thereby providing identification of daughter peptides and parent peptides. Additive measures include peak height, elution time, accurate mass, and combinations of the additive measures. Identification of daughter peptides and/or parent peptides and ultimately proteins in a sample includes comparing elution profiles for daughter peptides and parent peptides as a function of time with their corresponding accurate masses. Identification of protein in the sample, including daughter peptides and/or parent peptides can further include correlating additive measures for peak elution times for daughter peptides with peak elution times for corresponding parent peptides, thereby profiling same. Correlating additive measures such as peak elution times for daughter peptides and for corresponding parent peptides can be done using suitable algorithms. Predictions for peak elution times for parent peptides can be made using an artificial neural network. The artificial neural network yields probabilities for which parent peptides will be observed in the separations process. The present invention may be embodied in many different forms. For the purpose of promoting an understanding of the principles of the invention, reference will now be made to embodiments illustrated in the accompanying drawings, and specific language will be used to describe the same, in which like numerals in different figures represent the same structures or elements. It will nevertheless be understood that no limitation in scope of the invention is thereby intended. Any alterations and further modifications in the described embodiments, and any further applications of the principles of the invention as described herein are contemplated as would normally occur to one skilled in the art to which the invention relates. This abstract is neither intended to define the invention of the application, which is measured by the claims, nor is it intended to be limiting as to the scope of the invention in any way.

### BRIEF DESCRIPTION OF THE DRAWINGS

FIG. 1 presents a flow chart showing exemplary steps for conducting in-solution fragmentation, according to an embodiment of the process of the invention.

FIG. 2 illustrates exemplary stages of an in-solution fragmentation system of an online design that provides for identification of peptides of a sample protein, according to one embodiment of the invention.

FIG. 3 illustrates exemplary components of the in-solution fragmentation system of FIG. 2.

FIG. 4 illustrates an in-solution fragmentation system of a lab-on-a-chip design, according to an embodiment of the invention.

FIG. 5 illustrates exemplary stages of an in-solution fragmentation system of an offline design that provides for identification of peptides of a sample protein, according to yet another embodiment of the invention.

FIG. 6 presents distributions of peptides as a function of molecular weight obtained by cleavage of Homo sapiens proteins by different chemicals and enzymes.

FIG. 7 is a plot showing percentage of unique Homo sapiens peptides obtained as a function of molecular weight from in-silico analysis using various filtering criteria.

FIG. 8a depicts parent peptides (SEQ. ID. NOS: 1-16) obtained from in-solution fragmentation of Homo sapiens proteins taken from an in-silico database.

FIG. 8b depicts daughter parent peptides (SEQ. ID. NOS: 1749) obtained from in-solution fragmentation of parent peptides of FIG. 8a.

FIG. 9 shows amino acid sequences of a Carassin parent peptide (SEQ. ID. NO: 50) and three daughter peptides (SEQ. ID. NOS: 51-53) obtained from in-solution fragmentation of the Carassin parent peptide with trypsin.

FIG. 10a plots reverse phase gradient data and mirror gradient data for HPLC separation of a Carassin parent peptide (SEQ. ID. NO: 50) and three Carassin daughter peptides (SEQ. ID. NOS: 51-53) obtained from in-solution fragmentation of a Carassin protein respectively as a function of elution time.

FIG. 10b presents mass data (m/z) and elution data for the Carassin parent peptide (SEQ. ID. NO: 50) of FIG. 10a with three associated daughter peptides (SEQ. ID. NOS: 51-53) provided from in-solution fragmentation of the parent Carassin peptide.

### DETAILED DESCRIPTION OF THE INVENTION

The present invention is a system and process for fragmenting proteins in solution (so-called “in solution” fragmentation) that yields peptides of a size that, in conjunction with mass analysis, provide sufficient mass and structural information to improve accuracy and confidence in identifying peptides and eventually proteins in a sample. The invention finds application in proteomics analyses, e.g., for identification of complex proteins in protein mixtures. Fragmentation or cleavage of intact proteins in solution yields parent peptides of a preselected size or chain length. Further digestion of parent peptides yields daughter peptides of a still shorter chain length and size or molecular weight. Mass analysis data, and any allied separations data, of both parent and corresponding daughter peptides permit identification of proteins in the sample. The following terms are used herein. “In-solution fragmentation” means fragmentation (digestion) of a protein or polypeptide within a solution or liquid that breaks proteins or polypeptides in a sample into smaller parent peptides and further breaks parent peptides into smaller daughter peptides. In-solution fragmentation contrasts with fragmentation that occurs, e.g., in the gas phase of a mass spectrometer. In-solution fragmentation also contrasts with single or one-phase digestions, which are typically done offline, in which proteins and polypeptides in a sample are digested into parent peptides. The term “parent peptides” refers to peptides of a preselected size (e.g., molecular weight or length of the carbon backbone) that result from fragmentation or digestion of intact proteins and polypeptides in a sample. “Daughter peptides” refers to peptides that result from fragmentation or digestion of parent peptides. “Separations” as used herein means any process or device that physically separates parent peptides or daughter peptides into individual peptides or groups of peptides having like properties. Separations properties include, but are not limited to, e.g., molecular mass, size, carbon number, amino acid content, retention time, elution time, isoelectric point (pi), and like properties. “Online” means any process step or device that is integrated with, or conducted in combination with, other process steps, devices, and/or components of analysis systems or processes described herein. “Offline” means any process step or device that is conducted, or operated, outside of, or separate from, otherwise integrated components of an analysis system or process.

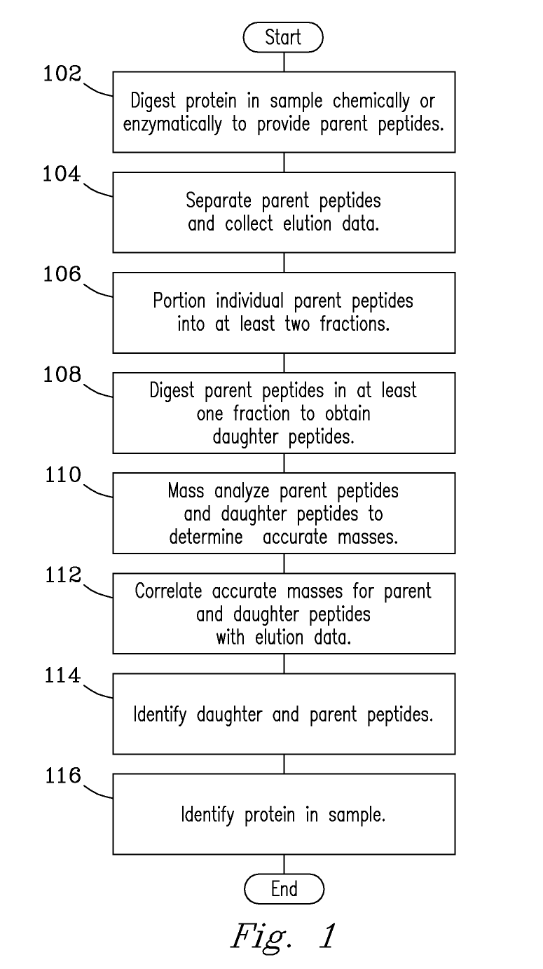

FIG. 1 is a flow chart showing exemplary steps for conducting “in-solution” fragmentation and analysis, according to a preferred embodiment of the process of the invention. [START]. In one step 102, proteins and/or polypeptides in a sample are fractionated (digested) into parent peptides of a preselected size. Digestion may be accomplished enzymatically and/or chemically, offline or online. In another step 104, parent peptides are separated into individual parent peptides or groups of parent peptides, e.g., in a liquid chromatography column or a separations method, and elution data including, e.g., retention time data, elution time data, migration time data, isoelectric point data, and/or other elution data, are collected. Elution data provide specific elution profiles for each parent peptide. In yet another step 106, individual parent peptides separated in the separations process are portioned into at least two fractions for further processing and/or analysis. In another step 108, individual parent peptides in at least one fraction are digested in succession to obtain daughter peptides. Here, digestion is preferably orthogonal, i.e., performed using an enzyme different from that used in the first fractionation step (102) to provide different structural information for identification of both the daughter peptides and the parent peptide from which the daughters are derived. Individual parent peptides portioned into a second fraction in succession remain undigested (i.e., as intact peptides) for further processing and/or analysis. In another step 110, individual parent peptides and associated daughter peptides in respective first and second fractions are analyzed in a mass analyzer or spectrometer to obtain accurate mass data by which to identify the individual parent peptides and the daughter peptides in respective fractions. Parent peptides and associated daughter peptides may be analyzed separately in a single mass analyzer or concurrently in separate mass analyzers. In another step 112, mass data acquired for both parent peptides and daughter peptides that includes, but is not limited to, e.g., ion spectra, accurate masses, m/z, intensities, abundances, and other mass data are analyzed. Mass data for parent peptides and daughter peptides may be further correlated with elution data collected previously in the separations step (see step 106) for parent peptides, as described further herein. In still yet another step 114, parent and daughter peptides are identified. In another step 116, proteins and/or polypeptides in the original sample are identified, e.g., using: sequence information obtained for both parent and daughter peptides; mass data; elution data; and other correlation information. [END].

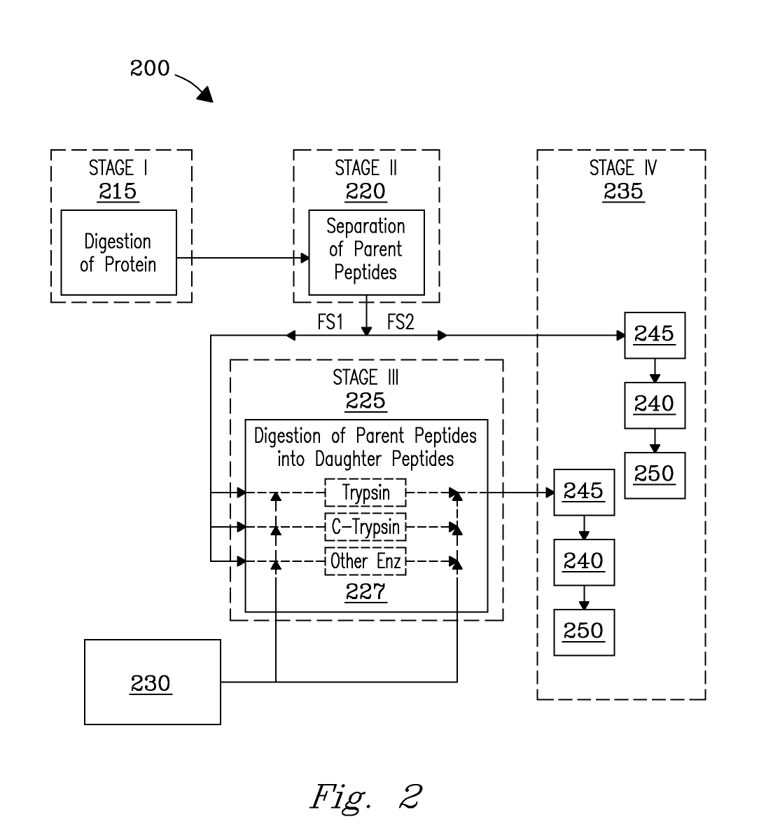

FIG. 2 illustrates an “in-solution” fragmentation system 200 of an online operation design, according to an embodiment of the invention. In the figure, system 200 includes: a first digestion (fragmentation) stage 215 (Stage I), a separations stage 220 (Stage II), a 2^{nd} digestion stage 225 (Stage III), and an analysis stage 235 (Stage IV). The system is suitable for analysis of proteins and/or polypeptides, e.g., in protein mixtures. In digestion stage 215 (Stage I), intact proteins or polypeptides present in a sample are fragmented (digested) “in-solution” to yield parent peptides. Fragmentation in stage 215 (Stage I) can be conducted chemically or enzymatically. Enzymatic digestion of proteins, polypeptides, and peptides in stage 215 (Stage I) is preferably accomplished using endopeptidases including, but not limited to, e.g., Lys-C, Asp-N, Glu-C, and like peptidases. Size of parent peptides is not limited. Enzymes used in conjunction with the invention may be of an immobilized (e.g., columnized) form suitable for online operation, or of a free form suitable for offline operations. Choice of enzymes is not intended to limited to exemplary enzymes described herein. Chemical digestion (Fragmentation) of proteins and polypeptides in stage 215 can be effected using any of a variety of chemical digestion reagents known in the proteomics art, including, e.g., cyanogen bromide (Cyan-Br), hydrochloric acid (HCl), trifluoroacetic acid (TFA), formic acid, and like chemical reagents. TFA, for example, chemically cleaves proteins at the C-terminal end of aspartic acid (Asp, or D) residues. Cyan-Br chemically cleaves proteins on the carboxyl side of methionine (Met, or M) residues. In Stage I, in-solution fragmentation cleaves intact proteins and polypeptides and provides parent peptides. Parent peptides are preferably of a size defined by a molecular weight in the range from about 1,000 Daltons to about 10,000 Daltons, but size is not intended to be limited thereto. More particularly, parent peptides are of a size defined by a molecular weight in the range from about 1,000 Daltons to about 6,000 Daltons. Most preferably, parent peptides are of a size defined by a molecular weight in the range from about 2,500 Daltons to about 6,000 Daltons. Peptides generated in fragmentation stage 215 (Stage I) are subsequently provided to a separations stage 220 (Stage II). In the separations stage, parent peptides from fragmentation stage 215 are physically separated. Separation of parent peptides is achieved using separations methods and devices known to those of skill in the chromatographic arts, including, e.g., Liquid Chromatography (LC). LC techniques include, but are not limited to, e.g., Normal Phase LC, Reversed Phase LC (RPLC), Strong-Cation Exchange (SCX) LC, 2-D LC, High-Pressure LC (HPLC) and like separations methods. Separations can also be achieved using, e.g., Electrophoresis, Capillary Electrophoresis (CE), Dielectrophoresis (DEP), Capillary Isoelectric Focusing, Gel separations in one or more dimensions, including, e.g., 2-D gel, Sodium Dodecyl Sulfate Polyacrylamide Gel Electrophoresis (SDS-PAGE); high-efficiency multidimensional separations, microseparations, microcolumn separations and like separation operations and devices. Separations can also be effected using LC columns in concert with stationary phases described herein, e.g., for online operation. In other embodiments, separations can be employed in conjunction with lab-on-a-chip processes and devices, microseparations processes and devices, and microcolumn separations configurations. No limitations are intended by the exemplary embodiments described herein. For example, as will be appreciated by those of skill in the art, any means of liquid-based and gel-based separations can be utilized in conjunction with the invention. As such, all process configurations and devices and as will be contemplated or implemented by those of skill in the art in view of the disclosure are within the scope of the invention. In online operation, separation of parent peptides in a liquid stream provides a unique elution profile, including data, e.g., for retention time, elution time, migration time, isoelectric point (pi), and/or other related and/or like properties for each eluting parent peptide, which data may be aligned and/or correlated with accurate mass data provided in analysis stage (Stage IV), described further herein. In cases of co-eluting parent peptides, deconvolution can be employed to simplify analysis, e.g., as detailed by Chakraborty et al. (Rapid Commun. Mass Spectrom. 2007, 21, 730-744), incorporated herein by reference. In the figure, the liquid stream containing each individual parent peptide, or groups of co-eluting parent peptides, separated in the separations stage (Stage II) is subsequently split following separation into at least two independent fluid streams, e.g., a first fluid stream (FS1) and a second fluid stream (FS2) that contains a portion or quantity of the parent peptide. While two streams, (FS1) and (FS2), are illustrated in the figure, number of streams is not limited. For example, multiple and independent liquid streams containing a quantity of individual (parent) peptides or co-eluting peptides separated in succession (e.g., as they elute) from an earlier stage may be used for conducting various analyses of interest, whether online or offline. At least one stream (FS1) containing a quantity of individual parent peptide separated in time is provided in succession to digestion stage 225 (Stage III) for further processing. At least one other (e.g., second) stream (FS2) containing a quantity of (intact) parent peptides separated in time is introduced in succession from separations stage 220 (Stage II) directly to analysis stage 235 (Stage IV), described further herein. In digestion stage 225 (Stage II), parent peptides in stream (FS1) are introduced in succession to Stage III and digested enzymatically with a suitable enzyme. Enzymes include, but are not limited to, e.g., trypsin, chymotrypsin, pepsin, and like proteases. Trypsin, for example, cleaves peptides between Lysine (Lys, or K) and Arginine (Arg, or R) residues. Chymotrypsin cleaves peptides between Phenylalanine (Phe or F), Tyrosine (Tyr or Y), Tryptophan (Trp or W), and to a lesser extent, between Methionine (Met or M) and Leucine (Leu or L) residues. Another suitable enzyme for digestion stage 225 is pepsin, which can cleave at Phenylalanine (Phe or F), Tyrosine (Tyr or Y), Tryptophan (Trp or W) and Leucine (Leu or L) residues. Further, because trypsin and chymotrypsin function at the same pH requirements, trypsin and chymotrypsin can be used in tandem. Enzymes selected for use in stage 225 (Stage II) are preferably orthogonal to (different than) those chosen for use in Stage 215 (Stage I) in order to provide daughter peptides with different structural information by which to identify the intact protein or polypeptide. Enzymes can be selected from different enzyme classes or can include different enzymes from within the same enzyme class. Suitable enzymes effect site-specific and/or target-specific cleavages and provide daughter peptides that have useful structural information. All enzymes contemplated by those of skill in the proteomics art for accomplishing enzymatic digestion and fractionation in view of the disclosure are encompassed hereby. Digestion of parent peptides introduced to stream (FS1) yields daughter peptides and/or fragments of a size defined by a molecular weight in the range from about 300 Daltons to about 6,000 Daltons. More particularly, molecular weight is about 1,500 Daltons, but is not limited thereto. Enzymatic digestion for stream (FS1) is preferably accomplished online in conjunction with immobilized enzymes capable of being used and reused in multiple analyses over time. Digestion of parent peptides in stream FS1 to generate daughter peptides is preferably rapid in order to provide a time scale that matches with the movement and analysis of intact parent peptides in another stream (FS2). Digestion of peptides online in stream FS1 is preferably done in a time of less than or equal to about 120 seconds. More preferably, digestion is effected in a time that is below about 60 seconds. Most preferably, digestion is effected in a time that is less than or equal to about seconds. Digestion time offline is not limited. By way of Illustration, in a non-limiting and exemplary configuration, the enzyme column containing immobilized enzymes can have a length of between about 1 cm and about 5 cm through which stream (SF1) flows. Flow path for second stream (SF2) can be of a tailored length or modulated so that the second stream (SF2) arrives at the same time as the first stream (SF1) to the mass analyzer. Time of arrival of streams SF1 and SF2 and/or mass analysis times for parent peptides, daughter peptides, and combinations of peptides are not critical, as alignment and correlation of various analysis times with elution data can still be performed. Speed of enzymatic digestion can also be modulated by the length and/or the inner diameter (I.D.) of the digestion column or digestion reactor and/or the density of the immobilized enzymes. Flow rates will further depend on the ID of the separations (i.e., chromatographic) capillary. In general, linear flow velocities will be within a preselected and narrow range (e.g., 1-4 μL/min in a 150 μm I.D. column) in order to achieve optimum chromatographic separation of parent peptides. The matching and alignment of data obtained by simultaneous mass analysis of peptides in streams (FS1) and (FS2) permits accurate mass data for both parent peptides and daughter peptides to be correlated. For example, analysis of parent peptides in a first stream (FS1) can be accomplished simultaneously with the digestion and analysis undertaken for daughter peptides in a second stream (FS2). Alternatively, mass analysis of first stream (FS1) can be performed serially with second stream (FS2). For example, one peptide in the first stream would be analyzed followed by analysis of one peptide in the other stream. In another process, mass analysis of first stream (FS1) and second stream (FS2) can be performed consecutively. Here, all the peptides in the first stream would be completely analyzed in succession, followed by analysis of all the peptides in the other stream in succession. In the figure, stream (FS1) introduced to stage 225 (Stage II) may be digested in one or more enzyme pathways simultaneously, serially in a single pathway, or in one or more digestion pathways, e.g., in conjunction with an enzyme column 227 of immobilized enzymes, described further herein. For example, digestion pathways may contain not only a single enzyme but several enzymes. Alternatively, the same liquid stream can pass first from, e.g., a trypsin digestion pathway to, e.g., a chymotrypsin digestion pathway, as well as additional enzyme digestion pathways. All enzymatic pathway configurations and mass analysis configurations as will be envisioned by those of skill in the art in view of the disclosure are within the scope of the disclosure. As with fragmentation provided in Stage I (i.e., a first digestion), digestion in stage 225 (i.e., a second digestion) can also be conducted chemically, as previously described herein. Thus, no limitations are intended. As further illustrated in the figure, either prior to, or immediately following enzymatic digestion in stage 225, any of a variety of reagents or solvents including, but not limited to, e.g., water, acetonitrile, ammonium acetate, ammonium formate, formic acid, other acids, and buffers can be optionally introduced to stream (FS1), e.g., from a reagent reservoir 230, e.g., to adjust pH or to optimize digestion. No denaturing agents are expected to be required for online digestion, as only peptides, not proteins, are digested in this stage. Following digestion in Stage III, daughter peptides in stream FS1 are introduced in succession to stage 235 (Stage IV) for mass analysis with a suitable mass analyzer 240. Parent or daughter peptides are preferably analyzed in a TOF mass analyzer, providing accurate mass data (e.g., m/z) for identification of the parent peptides in the selected stream, but choice of analyzer is not limited. The mass analyzer or spectrometer selected for analysis will depend on the desired end result including, e.g., post-translational identifications, de-novo sequencing, identification of non-modified peptides, protein identifications of known proteome organisms, etc.), and the complexity of the proteomic sample to be analyzed, as will be understood by those of skill in art. In analysis stage 235, mass analysis of daughter peptides in stream FS1 provides accurate mass data (e.g., m/z) and times by which to identify daughter peptides in the selected stream. Parent peptides introduced to analysis stage 235 in stream (FS2) from separations stage 220 are also analyzed in conjunction with an MS spectrometer or mass analyzer, providing accurate mass data and information for each parent peptide introduced in succession to the fluid stream for analysis. Mass data obtained for peptides in each stream may be correlated with the other to identify daughter and parent peptides. Elution data provided from separation of the parent peptides may also be included in the analysis. In alternate operations, stream (FS1) emerging from digestion stage 225 (Stage III) containing daughter peptides, and stream (FS2) emerging from separations stage 220 (Stage II) containing parent peptides, can be separately analyzed in stage 235 (Stage IV) in conjunction with a single MS analyzer, e.g., by quickly alternating from one stream to another. In other operations, each of streams (FS1) and (FS2) is analyzed separately but concurrently in separate MS analyzers, e.g., in a dual, split stream MS analysis system or equivalent, e.g., in conjunction with a dual channel ion funnel. Streams containing daughter and parent peptides are preferably electrosprayed into the MS analyzer, but approach is not limited thereto. As will be understood by those of skill in the MS art, streams (FS1) and (FS2) can be electrosprayed using a single electrospray emitter into a single MS analyzer or electrosprayed using separate electrospray emitters into the same or separate MS analyzers. No limitations are intended. All configurations as will be contemplated by those of skill in the art in view of the disclosure are within the scope of the invention. MS analyses of individual parent peptides and daughter peptides in respective process streams (FS1) and (FS2) provide high-resolution spectra and accurate mass data by which to identify daughter peptides and parent peptides, or that narrow the likely possibilities for identification of same. Accurate mass data for parent and daughter peptides can be further correlated on, e.g., identical time scales, with separations data (e.g., retention times, isoelectric point data, and like separations data) acquired for parent peptides in separations stage 220 (Stage II) that provides for alignment of data for parent and daughter peptides for identification of same, as described further herein. Correlations involving both mass accuracy data and elution data for individual parent and daughter peptides as described herein provide for identification of individual parent and daughter peptides without need for conventional MS/MS fragmentation and analysis. As will be understood by those of skill in the art, isoelectric point (pi) data can require additional isoelectric point separations following digestion (e.g., with Lys-C) in Stage 215 (Stage I) prior to separations in Stage 220 (Stage II). Thus, no limitations in process steps are implied by description of the exemplary stages herein. In another embodiment of system 200, the 2^{nd} digestion performed in stage 225 (Stage III) can be conducted partially, or turned on and off as needed for rapid control of the process. Partial digestion of individual parent peptides or groups of parent peptides can be achieved, e.g., by control of process parameters including, but not limited to, e.g., time of digestion, density of immobilized enzymes, temperature, addition of organic modifiers, or other process parameters such that digestion of parent peptides to daughter peptides is selective controlled. For example, switching digestion on and off online can be achieved by introducing rapid changes to the organic solvents through the mirror gradient or by adding other modifiers that will create a momentary pause (e.g., on the order of seconds) in digestion. In this way, e.g., parent peptides can be digested for a period of time (e.g., 2 sec.) followed by a period of time (e.g., another 2 sec.) with no digestion. In this way, parent and daughter peptides, or alternatively a higher then lower ratio of daughter to parent, reach the detector. And, as described, two process flow streams are not required. Further, control of the yield of daughter peptides from each 2^{nd} digestion step is not mandatory, although digestion of about half of each parent peptide is preferred in a single stream process. Thus, no limitations are intended. All processing conditions and configurations as will be contemplated by those of skill in the art in view of the disclosure are within the scope of the invention.

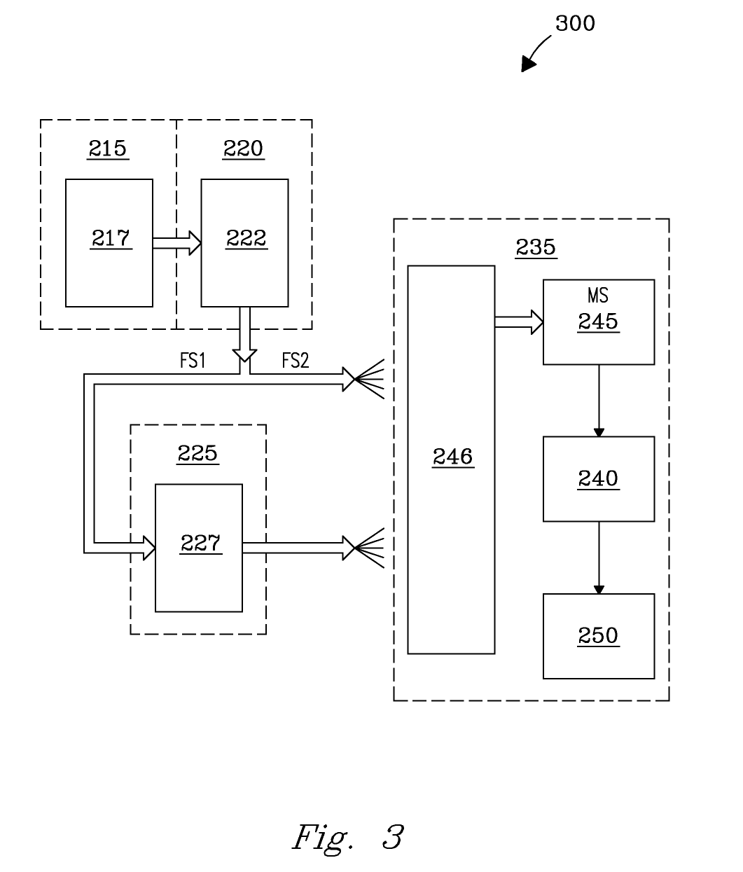

FIG. 3 illustrates an exemplary configuration 300 of an in-solution fragmentation system of FIG. 2 for online operation. In the figure, a first digestion of sample proteins and/or polypeptides, e.g., in protein mixtures, is conducted in solution in digestion stage 215 (Stage I) in, e.g., one or more digestion vessels 217 to yield parent peptides. Digestion vessels are not limited. Exemplary vessels include milliliter volume containers and tubes available commercially (Eppendorf Scientific, Hamburg, Germany). Fragmentation can be conducted chemically or enzymatically. Here, enzymatic digestion of proteins and/or polypeptides is preferably accomplished using endopeptidases including, but not limited to, e.g., Lys-C, Asp-N, Glu-C, and like peptidases. Parent peptides obtained in digestion stage 215 (Stage I) are subsequently provided to separations stage 220 (Stage II) where the parent peptides are physically separated. In the instant operation, separation is achieved using a C18 column 222 and stationary phase available commercially, which provides elution data including, but not limited to, e.g., retention time, isoelectric points, and like separations or elution data. In the figure, individual peptides or groups of peptides obtained from the separations column are portioned into at least two fluid streams, FS1 and FS2. Fluid stream FS1 containing a quantity of individual parent peptides separated in time is provided in succession to digestion stage 225 (Stage II), where the parent peptides are digested in a second digestion step in solution. In one embodiment, digestion is preferably conducted with an enzyme column 227 configured with an immobilized enzyme. Immobilization of enzymes is detailed, e.g., by Sakai-Kato et al. (Analytical Chemistry 2002, 74, (13), pgs. 2943-2949). Enzymes include, but are not limited to, e.g., trypsin, chymotrypsin, pepsin, and like proteases. As described herein, enzymes are preferably selected that are orthogonal to those employed in digestion stage 215, In the figure, intact parent peptides separated in time into fluid stream FS2 are introduced in succession from separations stage 225 (Stage II) directly to analysis stage 235 (Stage IV). Daughter peptides introduced in time into fluid stream FS1 from digestion stage 225 are introduced to analysis stage 235 (Stage IV) simultaneously with fluid stream FS2. In the figure, a dual-channel ion funnel 246, detailed, e.g., by Tang et al. (Analytical Chemistry, Vol. 74, Issue 20, pg. 5431-5437) acts as an interface to electrospray emitters 245 and MS analyzer 240. In the instant configuration, streams FS1 and FS2 are electrosprayed using separate electrospray emitters 245 into a single MS analyzer 240. Here, parent and daughter peptides are preferably analyzed in a TOF mass analyzer 240. MS analyses of individual parent peptides and daughter peptides in respective process streams (FS1) and (FS2) provide high-resolution spectra and accurate mass data by which to identify daughter peptides and parent peptides, or that narrow the likely possibilities for identification of same. Accurate mass data for parent and daughter peptides can be further correlated on, e.g., identical time scales, with separations data (e.g., retention times, isoelectric point data, and like separations data) acquired for parent peptides in separations stage 220 (Stage II). Correlations involving both mass accuracy data and elution data for individual parent and daughter peptides as described herein provides for identification of individual parent and daughter peptides without need for conventional MS/MS fragmentation and analysis.

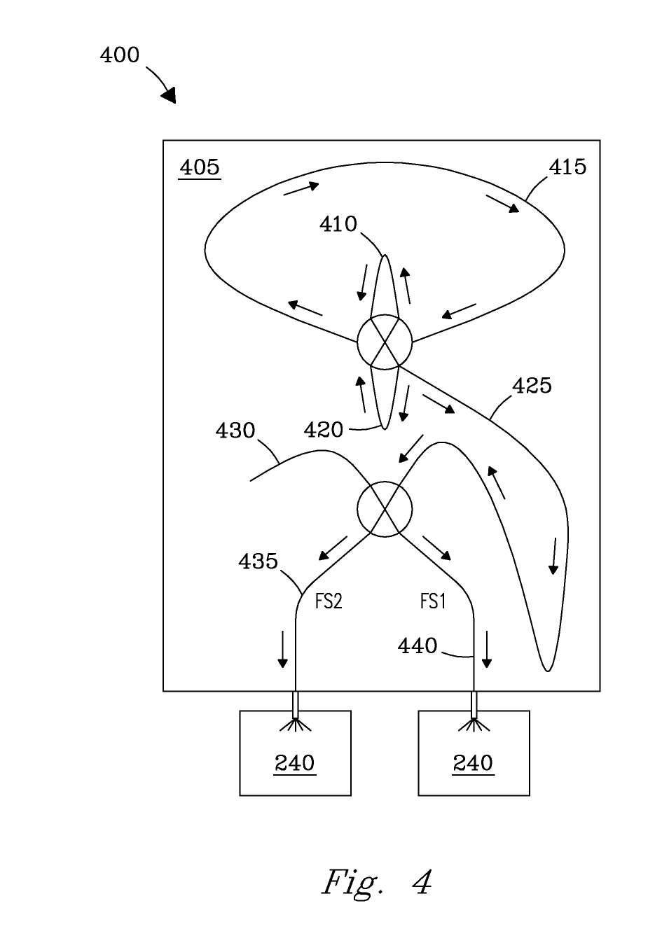

FIG. 4 illustrates an in-solution fragmentation system 400 of an exemplary lab-on-a-chip design, according to an embodiment of the invention. Lab-on-a-chip is a term for devices that integrate multiple laboratory functions on a single chip 405. In the figure, chip 405 has dimensions that range from square millimeters to square centimeters in size and is capable of handling extremely small fluid volumes, e.g., picoliters or less. In the figure, 2 trapping columns are illustrated, e.g., a strong cation exchange (SCX) enrichment column 410 and an enrichment column before Reverse Phase (RP) 420. Two separation columns are also shown, e.g., an SCX separations column 415 and a Reverse Phase separations column 425. A reverse (mirror) gradient column 430 is also shown. At the end of the flow path, prior to introduction into the MS 240 for analysis, a post column digestion line 435 is included that contains immobilized enzyme, which provides digestion of separated parent peptides in one flow path prior to introduction into the MS 240. Trapping columns, separations columns, and the digestion column are linked by way of microfluidic flow lines that provide the necessary flow paths to obtain the desired fragmentation. As will be appreciated by those of skill in the art, it is possible to simplify peptide mixtures by separating them (online or offline) by two or more orthogonal chromatographic/electrokinetic techniques, e.g., in two-dimensional or multidimensional chromatography. Here, proteins are digested to obtain parent peptides. The parent peptides are separated, e.g., using a strong cation exchange (SCX) column 415 or isoelectric focusing column, or via other separations techniques and columns known in the art; eluted parent peptides are either: A) collected offline and injected into the SCX column loop for separation, or B) are directed online to the reversed phase (RP) chromatography (RPC) column 420. RPC is preferred as the last peptide separation step just prior to mass analysis due to the high peak capacities obtained. In the figure, the lab-on-a-chip configuration provides both a two-dimensional peptide separation, as well as the necessary fluid components to provide desired fragmentation in solution. In one exemplary mode of operation, a sample protein of interest can be digested offline, e.g., using chemical digestion (e.g., with formic acid) which will cleave proteins at aspartic acid residues, generating multiple parent peptides. Parent peptides are subsequently injected into the lab-on-a-chip device and trapped in the SCX enrichment column 410 using a mobile phase containing an aqueous solution of ammonium formate (e.g., 5 mM at pH 3). Once parent peptides are loaded onto the column, ammonium formate (˜several microliters, 10 mM, at pH 3) is introduced, which elutes the parent peptides through SCX column 415 and introduces them into reversed phase enrichment column 420. Injection of water (˜several microliters) desalts the peptides. A reversed phase gradient is then initiated that over time is changes the high aqueous acidic pH mobile phase to a high organic (i.e. methanol or acetonitrile) mobile phase, which provides further separation of the parent peptides. Parent peptides with different retention times are then eluted. Eluent carrying the separated parent peptides is then split post-column into two fluid streams, FS1 and FS2. A first stream FS1 passes through an empty flow line. A second stream FS2 containing parent peptides is subjected to online digestion, e.g., through a flow line that contains, e.g., an immobilized trypsin-chymotrypsin enzyme combination. Immobilization of enzymes in microfluidic devices is detailed, e.g., by Peterson et al. (Analytical Chemistry 2002, 74, (16), 4081-4088). Both streams proceed to different electrospray emitters (FIG. 2) where they are electrosprayed, e.g., through a dual channel ion funnel (FIG. 2), which allows each electrosprayed stream to be alternatively mass analyzed by a mass analyzer 240 in a time scale on the order of microseconds. Each stream containing parent and daughter peptides that is directed to the mass analyzer has an identical elution time because digestion of parent peptides in one stream occurs following separation of the parent peptides. A detector (FIG. 2) provides different mass signals for parent and daughter ions received in succession in each stream, thus differentiating daughter or parent peptides from other pairs of parent and daughter ions received subsequently. Specificity provided by the high mass accuracy of parent and daughter peptides, in combination with elution time data and information from separation of parent peptides, allows for identification of peptides at a high confidence level. One cycle is thus completed. Another injection (several microliters) of a higher ionic strength buffer (for example 20 mM of ammonium acetate at pH 3) is injected to the strong cation exchange column, which will also carry another quantity of parent peptides to reversed phase column as described previously. Approximately 10-20 similar cycles can be done, each time with an increase in ionic strength of the buffers that elutes more and more peptides from the strong cation exchange column to the reversed phase column with subsequent mass analysis. As will be appreciated by those of skill in the art, several variations of the present operation can be performed. For example, instead of splitting the flow stream post column, two chromatographic runs can be done. One stream can proceed without post-column digestion; the other stream can proceed with post-column digestion, which allows for alignment of the two chromatograms for parent and daughter peptides, respectively. Alignment is simplified by the fact that a number of parent peptides are not digested further (i.e., in post-column digestion) because they do not contain necessary residues for digestion to occur. Alternatively, parent peptides may not be completely digested and will be found in both chromatograms. Parent peptides can also be used as internal standards in order to align the two chromatograms. In another exemplary operation, the two post column fluid streams can be combined back into one flow path for introduction to a single electrospray emitter into a single mass analyzer. This eliminates need for two different electrospray emitters. This approach is presumed to be inferior to the former processes because of the additional challenges introduced to differentiate parent ions from daughter ions. The instant operation still retains the advantage that pairs of parent and daughter ions are separated from other pairs of parent and daughter ions eluted in time. No limitations in operation parameters are intended. All configurations as will be implemented by those of skill in the art in view of the disclosure are within the scope of the invention.

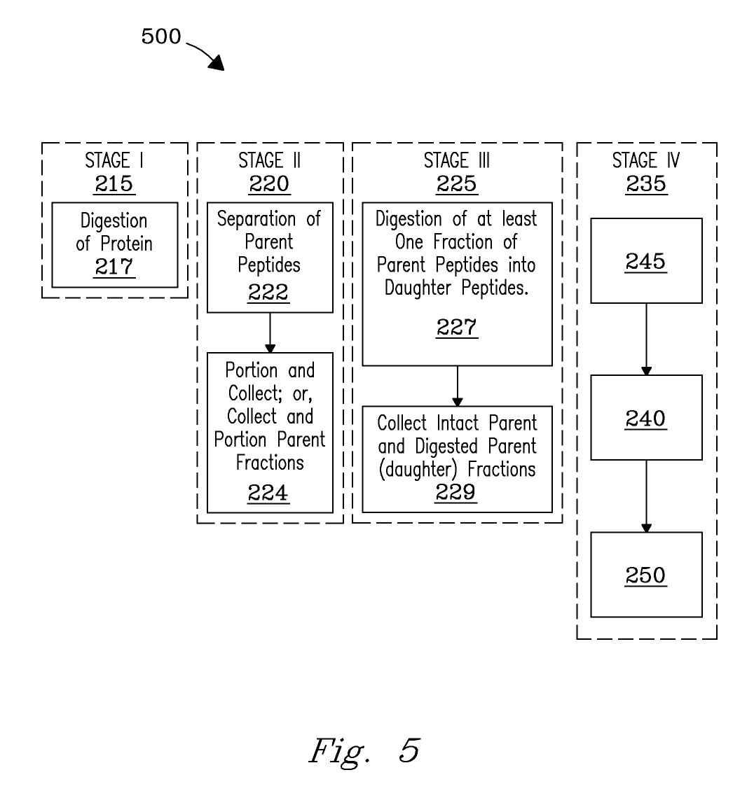

FIG. 5 illustrates an “in-solution” fragmentation system 500 of an offline design, according to an embodiment of the invention. In the figure, system 500 includes: a fragmentation (digestion) stage 215 (Stage I), a separations stage 220 (Stage II), a digestion stage 225 (Stage II), and an analysis stage 235 (Stage IV). The system is suitable for fragmentation and analysis of proteins and/or polypeptides, e.g., in protein mixtures. In digestion stage 215 (Stage I), intact proteins or polypeptides present in a sample are fragmented (digested) “in-solution” to yield parent peptides. Digestion of sample proteins is conducted, e.g., in one or more digestion vessels 217 to yield parent peptides, as described previously herein. Again, fragmentation (digestion) can be done enzymatically or chemically. Parent peptides are preferably of a size defined by a molecular weight in the range from about 1,000 Daltons to about 10,000 Daltons, but are not limited. Peptides generated in fragmentation stage 215 are subsequently provided to a separations stage 220 (Stage II). In separations stage, parent peptides are physically separated. Separation of parent peptides is effected using separations methods and devices described previously herein. Any liquid based separation method and device can be used in conjunction with the invention. As such, all process configurations and devices and as will be contemplated or implemented by those of skill in the art in view of the disclosure are within the scope of the invention. Here, separation is preferably achieved in a C18 column 222 as described previously herein, but is not limited. Separated parent peptides are collected and portioned. In one mode of operation, separated parent peptides are split or portioned into at least two streams, e.g., using a stream splitter and subsequently collected in a collection device 224 as the parent peptides elute. In an alternate operation, parent peptides are collected in a collection device 224 and then portioned into at least two fractions as they elute. Collection devices include, but are not limited to, e.g., well plates (e.g., 96 well plates, 394 well plates, and like devices), MALDI plates, and like collection devices. At least one fraction collected for each separated parent peptide is passed to digestion stage 225 (Stage III) where the parent peptide is digested into daughter peptides, as described previously herein, and subsequently passed to analysis stage 235. Digestion is preferably conducted with an enzyme column 227 configured with one or more immobilized enzymes, or multiple flow paths configured with respective enzyme columns containing one or more immobilized enzymes, but is not limited thereto. In offline operation, at least one intact parent fraction are collected for subsequent analysis along with the digested parent (i.e., daughter) fractions. Daughter peptides are collected in another collection device 229 for subsequent analysis. Intact (undigested) parent peptides in at least one fluid fraction are passed directly to analysis stage 235. In analysis stage 235, samples are individually mass analyzed. In the figure, a single mass analyzer 240 is shown. Samples containing either daughter peptides or intact parent peptides are infused, electrosprayed in an electrospray emitter 245 and introduced to analyzer 240, where they are detected by a mass detector 250.

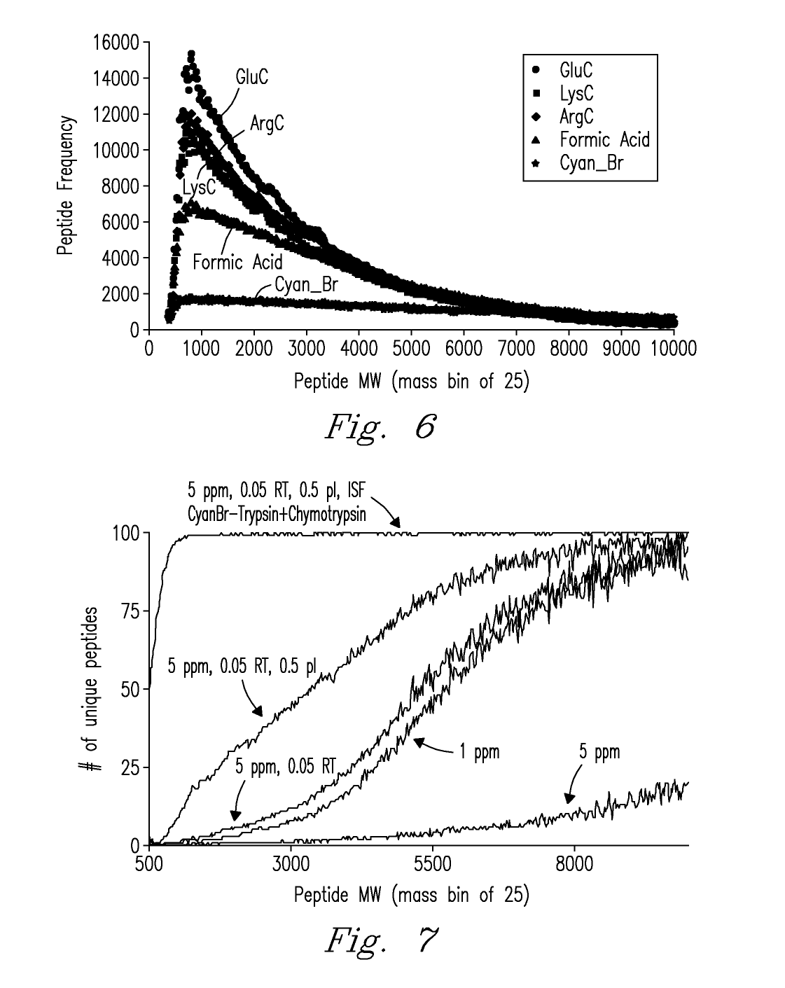

FIG. 6. is a plot showing frequency of peptides generated by various enzymes and chemical reagents in-silico. The figure shows which digestion methodologies provide higher molecular weight peptides on average. In the figure, digestion of proteins, polypeptides, and peptides in a first digestion stage (FIG. 2 and FIG. 5) is preferably accomplished using chemical or enzymatic digestions that generate parent peptides having relatively large molecular weights. Preferred weights are listed hereinabove (see discussion for FIG. 2). Large peptides are preferred as they are more unique, meaning there are fewer peptides from the same sample that will have an identical masses and retention times. Unique peptides also have a higher probability that they can be further digested in the second digestion stage into daughter peptides that will provide additional structural information. Small peptides (e.g., below 1,000 Daltons) do not provide additional structural information, generally. Cyan-Br is an excellent chemical reagent for cleavage of peptides, but is impractical and toxic. Formic acid digestion is a next best candidate of those digestion methodologies tested herein. Formic acid also is completely orthogonal to trypsin and chymotrypsin which are considered exemplary candidates for the second digestion for the generation of daughter peptides. In the figure, other suitable endopeptidases (enzymes) for enzymatic digestion are shown that include, but are not limited to, e.g., Lys-C, Asp-N, Glu-C, Arg-C, and the like. Lys-C, for example, cleaves proteins, polypeptides, and peptides at the C-terminus (i.e., free carboxyl group side of a peptide bond) between Lysine residues (Lys, or K) to free the (A.A.—Lys) peptides; Asp-N cleaves proteins, polypeptides, and peptides at the N-terminus (free amine side) between aspartic acid (Asp, or D) residues; Glu-C cleaves peptides between glutamic acid (Glu, or E) and aspartic acid (Asp or D) residues. Endopeptidases that cleave at only one specific residue along a peptide backbone provide on average larger peptides and thus simpler mixtures, than do proteases such as trypsin, chymotrypsin, and pepsin, which cleave at several residues. Trypsin, for example, cleaves proteins, polypeptides, and peptides between residues of both lysine and arginine (Arg, or R), yielding generally smaller peptides and thus more complex peptide mixtures. Chemical digestion of proteins, polypeptides and peptides in the first digestion stage is preferably accomplished using cyanobromide, formic acid, and/or acetic acid digestion. Cyanogen bromide cleaves proteins before methionine (Met or M), while formic acid and acetic acid cleave proteins before and after aspartic acid (Asp or D) residues.

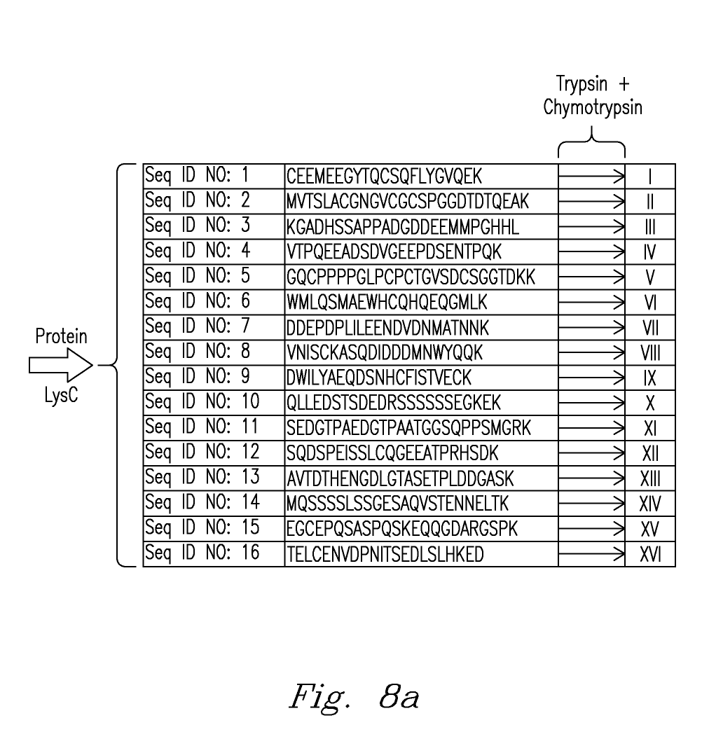

FIG. 7 is a plot showing number of unique peptides derived from in-silico digestion of Homo sapiens proteins and peptides as a function of peptide molecular weight (X-axis) and various filtering criteria including, e.g., mass accuracy (ppm), retention time (RT), isoelectric point (pi), and in-solution fragmentation (ISF). Unique peptides are defined as peptides that can be identified with high confidence under preselected analysis conditions. As shown in the figure, any combination of mass accuracy (e.g., with 1 ppm accuracy and 5 ppm accuracy), retention time (e.g., within ±5% of predicted retention time or 0.05 units) and isoelectric point (within ±0.5 pl units of the actual pl value) information does not provide sufficient peptide uniqueness (also termed specificity) to confidently identify peptides using the in silico database of human peptides. Specificity provided by various mass and elution parameters are detailed, e.g., by Norbeck et al. (J Am Soc Mass Spectrom 2005, 16, 1239-1249), incorporated herein in its entirety. As shown in the figure, by contrast, when in-silico digestion of human proteins and peptides is performed under theoretical in-solution fragmentation conditions (e.g., using Cyan-Br in a first digestion and Trypsin-Chymotrypsin in a second digestion)—in addition to other mass accuracy (e.g., 5 ppm mass accuracy) and elution parameters (e.g., +/−5% retention time prediction accuracy)—sufficient specificity is provided for peptides with a molecular weight (MW) greater than 1000 Daltons to be identified with confidence. In the figure, greater than 91% of peptides having a MW ≧1000 Daltons are unique, while greater than 99% of peptides with a MW ≧1500 are unique. Results demonstrate that in-solution fragmentation dramatically improves the ability to provide structural information by which to identify peptides. Use of retention time predictions and accurate prediction of peptide LC elution times for proteome analyses are detailed, e.g., by Petritis et al. (Analytical Chemistry, Vol. 75, Issue 5, pgs 1039-1048), Strittmatter et al (J. of Proteome Res., Vol. 3, Issue 4, pgs 760-769), and Petritis et al. (Analytical Chemistry, Vol. 78, Issue 14, pgs. 5026-5039), incorporated herein. Peptide isoelectric point predictions and uses are described, e.g., by Cargile et al. (J. Proteome Res., Vol. 3, Issue 1, pgs. 112-119) and Heller et al. (J. Proteome Res., Vol. 4, Issue 6, pgs. 2273-2282), incorporated herein.

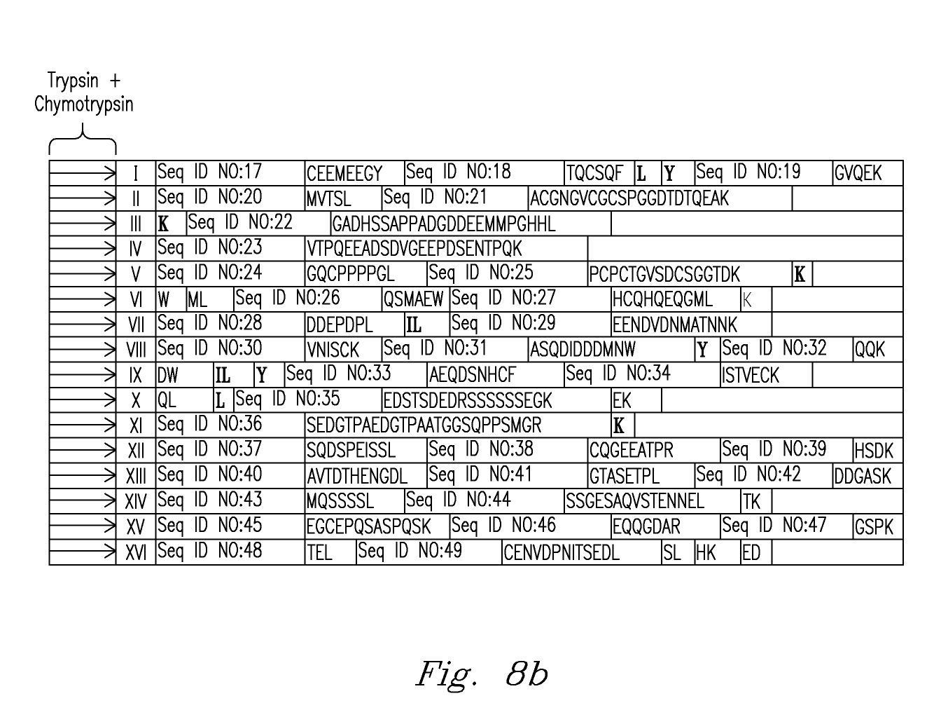

FIG. 8a depicts parent peptides (SEQ. ID. NOS: 1-16) obtained from in-solution fragmentation, using an exemplary enzyme, of Homo sapiens proteins taken from an in-silico database.

FIG. 8a presents a list of parent peptides (SEQ. ID. NOS: 1-16) obtained from in-silico digestion of human (Homo sapiens) proteins selected from an in-silico database under theoretical in-solution fragmentation conditions within a mass range of 50 ppm, i.e., from 2500.02321 Daltons to 2500.12747 Daltons. In the figure, Proteins were theoretically digested with Lys-C in a first in-solution digestion to obtain listed parent peptides, which were subsequently theoretically digested with a combination of trypsin and chymotrypsin in a second digestion. Following the first digestion with Lys-C, 16 peptides (SEQ. ID. NOS: 1-16) were obtained in the selected mass range. These parent peptides, if contained within a sample mixture, would typically co-elute. As such, they would not normally be distinguished based solely on accurate mass and time data from a single digestion in a standard separation and mass analysis process. Insufficient information would be available to identify these parent peptides and any sample proteins. This situation contrasts with the added information provided by in-solution fragmentation as follows. FIG. 8b depicts daughter parent peptides (SEQ. ID. NOS: 17-49) obtained from in-solution fragmentation of parent peptides of FIG. 8a using an exemplary enzyme combination (e.g., with trypsin-chymotrypsin). As shown in the figure, in-solution fragmentation provides 32 unique daughter peptides (SEQ. ID. NOS: 17-49) with a separation distance of at least 100 ppm that provide additional structural information by which to identify daughter peptides and parent peptides, or to narrow the list of possible daughter peptides and parent peptides in the sample. This example provides proof of concept of the in-solution fragmentation process for identification of sample peptides by generation of unique parent peptides and daughter peptides.

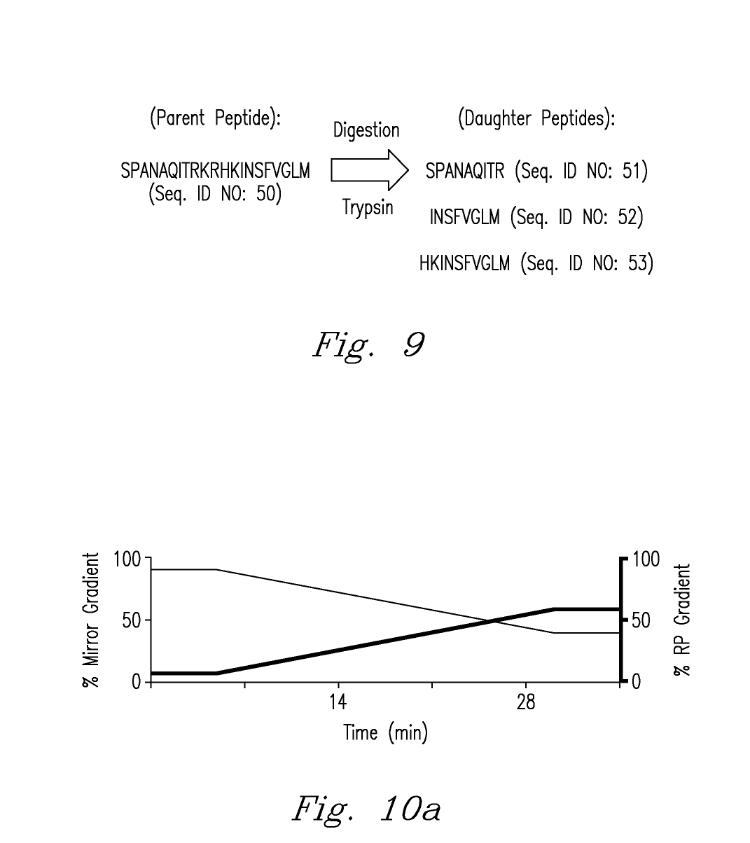

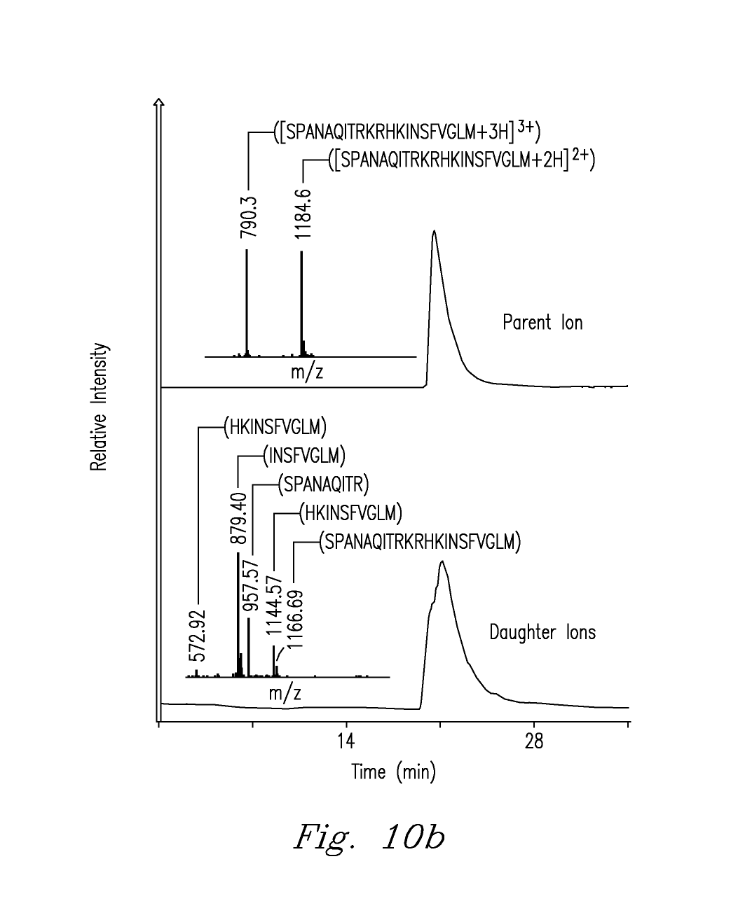

FIG. 9 is a schematic that demonstrates utility of in-solution fragmentation for analysis of sample proteins. In the figure, an amino acid sequence is presented of a representative Carassin parent peptide (SEQ. ID. NO: 50), with three unique Carassin daughter peptides (SEQ. ID. NOS: 51-53) obtained by the process of in-solution fragmentation of the Carassin parent peptide involving a second digestion with trypsin. Carassin peptide is a 21-amino acid tachykinin-related peptide originally isolated from goldfish brain. FIG. 10a plots reverse phase gradient data and mirror gradient data used for the separation of the Carassin parent peptide (SEQ. ID. NO: 50). The gradient elution profile is shown. In the reversed phase procedure, a non-polar stationary phase and a moderately polar aqueous mobile phase are used. A mobile phase composition is considered isocratic if the selected mobile phase composition remains unaltered during a separations procedure. The mobile phase may comprise of a single solvent or a pre-mixed mixture of different solvents. Under gradient elution reversed phase conditions, the stationary phase remains the same while the mobile phase composition changes over time from a more polar state to a less polar state. In the figure, mobile phase A has a composition of 95:5:0.1 [water:acetonitrile:formic acid]; mobile phase B has a composition of 5:95:0.1 [water:acetonitrile:formic acid]. In a mirror gradient experiment, the gradient elution has an opposite solvent composition to that used for the primary gradient elution. An additional chromatographic pump is used in order to generate the mirror gradient profile. The peptide separation is done under gradient elution acidic conditions, in which concentration of acetonitrile in the mobile elution phase varies over time. An inverse gradient is generated with an additional pump which keeps concentration of the acetonitrile constant, and, at the same time, modifies the pH to be compatible with the trypsin digestion (˜pH 8.2). Trypsin and chymotrypsin operate at optimum conditions that are not compatible with common reversed phase conditions. For example, enzymes can be denatured at high organic solvent concentrations and lose activity. Sudden changes in organic solvent can also stress enzymes and again drop activity. Under these conditions, recovery times can increase from minutes to hours. Optimum pH for these two enzymes is about pH=8 whereas peptide reversed phase separation takes place at a pH of 1.5 to about 3.5. At these pH values, enzyme activity is nearly zero. Use of a mirror gradient ensures that the concentration of organic solvent is held constant and at an acceptable limit for the enzymes to operate. At around 40% concentration, trypsin activity increases generally. Further, a mirror gradient is buffered so as to achieve a pH in the mobile phase of around pH=8, which is optimum for trypsin and chymotrypsin. As a result, trypsin works at optimum pH but at a constant concentration of acetonitrile. FIG. 10b shows a simplified proof of concept of the in-solution fragmentation process, demonstrated in conjunction with a Carassin parent peptide. The Carassin parent peptide (SEQ. ID. NO: 50) can be generated, e.g., by digestion of an intact protein, followed by separation of parent peptides followed both with online digestion and without online digestion with trypsin, followed by subsequent analysis, e.g., with an ion-trap mass spectrometer. In the figure, ion-trap mass data and elution data for the parent peptide are compared with data for the daughter peptides (SEQ. ID. NOS: 51-53). The upper chromatogram shows the double and triple charge of the Carassin parent peptide (SEQ. ID. NO: 50) without further online digestion. The lower chromatogram shows three unique daughter peptides (SEQ. ID. NOS: 51-53) obtained by online digestion of the parent peptide with trypsin. As can be seen, identical retention times are obtained for both parent and daughter peptides given that the daughter peptides are generated subsequent to the elution of the parent peptide. Accurate mass data for both parent and daughter peptides, as well as their respective retention times, significantly increases the specificity of the analysis (described previously in reference to FIG. 7). In the figure, low mass accuracy spectra were acquired. Correlation between the parent and daughter ions distinguished the Carassin parent peptide (SEQ. ID. NO: 50) at a high confidence level out of more than 500,000 in-silico generated Shewanella oneidensis peptides. The correlation also distinguished the peptide out of more than 5,000,000 in-silico generated Homo sapiens peptides. In the latter case, although the Expectation Value (E-value), a measure of statistical confidence, was >0.05, implying a less confident peptide identification, the peptide was selected as a first hit. The correlation was achieved using a MASCOT peptide fingerprinting approach, performed as follows: a) performed an in-silico digestion of the Shewanella oneidensis proteome (4198 proteins, file Shewanella_{—}2006-07-11.fasta) using Glu-C as the enzyme, which cleaves after aspartic acid (Asp, or D) and glutamic acid (Glu, or E) residues. Fragments were limited to those having a mass between 2360 Daltons and 2376 Daltons, given that the mass of the parent peptide was known. This yielded 2128 peptides. The sequence of the known Carassin parent peptide (SEQ. ID. NO: 50) was appended to the list of 2128 peptides in the selected mass range to define a list of 2129 “candidate” parent peptides. Peptides were loaded into MASCOT and a peptide mass fingerprint search was performed against the 2129 peptides using the m/z values for the three observed daughter peptides (SEQ. ID. NOS: 51-53) (879.4, 957.57, and 1144.57, respectively) shown in FIG. 10b with a match tolerance of ±2 Daltons. The search returned only one significant hit, i.e., the expected Carassin parent peptide SPANAQITRKRHKINSFVGLM (with mass 2367 Daltons). The MASCOT Mowse Score was 47; Expectation value (E-value) was 0.04. The next highest scoring parent peptide had a score of 15 and an E-value of 69. b) Next, an in-silico digestion of the Human proteome (61,225 proteins, file H_sapiens_IPI_{—}2006-08-22.fasta) was performed using Glu-C as the selected enzyme. Fragments were limited to those having a mass between 2360 Daltons and 2376 Daltons, which yielded 38,798 peptides. Redundant peptides were removed to give 18,468 unique peptides with masses in the selected range. The known Carassin parent peptide sequence was appended to the list of 18,468 peptide candidates to define a list of 18,469 candidate parent peptides. Candidate peptides were loaded into MASCOT and a peptide mass fingerprint search was performed using m/z values for the three observed Carassin daughter peptides (SEQ. ID. NOS: 51-53) (879.4, 957.57, and 1144.57, respectively) and a match tolerance of ±2 Daltons. The search returned no significant hits. However, the top scoring match was the expected parent peptide SPANAQITRKRHKINSFVGLM (with mass 2367 Daltons). Here, the MASCOT Mowse Score was 47; Expectation value was 0.34. The next highest scoring parent peptide had a score of 31 and an E-value of 14. This simplified example illustrates that the correlated parent ion/daughter ion approach provided for by in-solution fragmentation systems and processes described herein can significantly improve peptide identification confidence in proteomic analyses.

### CONCLUSIONS

The in-solution fragmentation systems and processes of the invention described herein provide parent peptides and associated daughter peptides. In-solution fragmentation of parent peptides is complete and avoids the undersampling and loss of structural information associated with gas-phase fragmentation. A unique identity can be assigned to the peptides due to the high specificity of the method which combines high mass accuracy of parent and daughter peptides along with elution data (e.g., retention time) derived from separations of the parent peptides. While the present invention has been described in reference to the preferred embodiments thereof, the invention is not limited thereto and may be embodied in many different forms. No limitation in scope of the invention is intended by the description of the preferred embodiments. All alterations and further modifications of the invention that will be undertaken by those of skill in the art in view of the description, including further applications of the principles of the invention, are within the scope of the invention.

We claim:

1. An in-solution fragmentation process, comprising the steps of:
digesting a protein or polypeptide in solution or in gel to obtain parent peptides;
separating said parent peptides to obtain individual parent peptides or groups of parent peptides;
portioning said individual parent peptides or said groups of parent peptides into at least two fractions that contain same; and
digesting said individual parent peptides or said groups of parent peptides in at least one of said at least two fractions in solution or in gel to obtain daughter peptides for same, said daughter peptides have a size that is less than or equal to said parent peptides.

2. The process of claim 1, wherein the step of digesting said protein in solution is performed at least partially with a chemical reagent.

3. The process of claim 2, wherein said chemical reagent includes a member selected from the group consisting of: cyanogen bromide, formic acid, acetic acid, and combinations thereof.

4. The process of claim 1, wherein the step of digesting said protein in solution is performed at least partially with an enzyme.

5. The process of claim 4, wherein said enzyme is an immobilized enzyme.

6. The process of claim 4, wherein said enzyme is an endopeptidase selected from the group consisting of: Lys-C, Asp-N, Glu-C, Arg-C, and combinations thereof.

7. The process of claim 1, wherein the step of separating said parent peptides includes a separations process or device that provides retention times for said individual parent peptides or groups of parent peptides.

8. The process of claim 7, wherein said separations process or device is a liquid chromatography separations process or device.

9. The process of claim 7, wherein said separations process or device includes a multiplate separations process or device.

10. The process of claim 7, wherein said separations process or device is a C18 separations process or device.

11. The process of claim 1, wherein the step of digesting said individual parent peptides or said groups of parent peptides in at least one of said at least two fractions includes a complete digestion of same.

12. The process of claim 11, wherein the step of digesting said individual parent peptides or said groups of parent peptides is accomplished in a time of less than 120 seconds.

13. The process of claim 11, wherein the step of digesting said individual parent peptides or said groups of parent peptides is accomplished in a time of less than or equal to 5 seconds.

14. The process of claim 1, wherein the step of digesting said individual parent peptides or said groups of parent peptides in at least one of said at least two fractions includes a partial digestion of same.

15. The process of claim 14, wherein the step of digesting said individual parent peptides or said groups of parent peptides is accomplished in a time of less than 60 seconds.

16. The process of claim 14, wherein the step of digesting said individual parent peptides or said groups of parent peptides is accomplished in a time of less than or equal to 5 seconds.

17. The process of claim 1, wherein the step of digesting said individual parent peptides or said groups of parent peptides in at least one of said at least two fractions is performed at least partially with an enzyme.

18. The process of claim 17, wherein said enzyme is an immobilized enzyme.

19. The process of claim 17, wherein said enzyme is an enzyme other than Lys-C, Asp-N, Glu-C, Arg-C.

20. The process of claim 17, wherein said enzyme is selected from the group consisting of: chymotrypsin, trypsin, pepsin, and combinations thereof.

21. The process of claim 1, wherein said process is conducted online or offline.

22. The process of claim 1, further comprising use of an artificial neural network process or device for prediction of retention times of parent peptides.

23. The process of claim 22, wherein said artificial neural network process or device provides for anticipating which of said parent peptides is observed during separation of same.

24. The process of claim 1, further comprising the step of mass analyzing said individual parent peptides or groups of parent peptides and said daughter peptides derived from same in a single mass analyzer.

25. The process of claim 1, further comprising the step of mass analyzing said individual parent peptides or groups of parent peptides and said daughter peptides derived from same simultaneously in separate mass analyzers.

26. The process of claim 25 wherein the step of mass analyzing said daughter peptides and said individual parent peptides or groups of parent peptides includes use of an electrospray emission process or a MALDI ionization process.

27. The process of claim 26, wherein the step of mass analyzing said daughter peptides and said individual parent peptides or groups of parent peptides includes use of a dual channel ion funnel.

28. The process of claim 1, wherein the step of mass analyzing said daughter peptides and said individual parent peptides or groups of parent peptides does not include a prior gas fragmentation step.

29. The process of claim 1, further comprising the step of identifying said protein.

30. The process of claim 29, wherein the step of identifying said protein includes correlating mass data and elution data for said parent peptides or said groups of parent peptides and said daughter peptides derived therefrom.

31. The process of claim 30, wherein the step of correlating said mass data and said elution data includes at least one parameter or measure selected from the group consisting of: accurate mass, retention time, isoelectric point, probability of peptide elution, and combinations thereof.

32. The process of claim 31, wherein the step of correlating said mass data and said elution data includes aligning time data from separations of said individual parent peptides or said groups of parent peptides and said daughter peptides.

33. The process of claim 30, wherein the step of correlating said mass data and said elution data provides for de novo sequencing of said protein.

34. The process of claim 1, wherein said process is performed with an on-chip process or on-chip device.

35. The process of claim 1, wherein one or more steps of said process are performed online.

36. The process of claim 1, wherein one or more steps of said process are performed offline.

37. The process of claim 1, wherein the process is performed in a microscale fluid process or microscale fluid device.

38. An in-solution fragmentation process, comprising the steps of:
digesting a protein in solution or in gel to obtain parent peptides;
separating said parent peptides to obtain individual parent peptides or groups of parent peptides;
digesting said individual parent peptides or said groups of parent peptides at least partially in solution or in gel to obtain at least a quantity of daughter peptides for same, said daughter peptides have a size that is smaller than said parent peptides.
